(19) United States
(12) Patent Application Publication
(10) Pub. No.: US 2026/0069764 A1
(43) Pub. Date: Mar. 12, 2026

(54) METHOD OF OPERATING A LARGE-VOLUME DELIVERY DEVICE

(71) Applicant: Sanofi-Aventis Deutschland GmbH, Frankfurt am Main (DE)

(72) Inventors: Marc Anderson, Marblehead, MA (US); Donald Ziniti, Cumberland, RI (US)

(21) Appl. No.: 18/826,782

(22) Filed: Sep. 6, 2024

(51) Int. Cl. A61M 5/142; A61M 5/145

(52) U.S. Cl. CPC A61M 5/14248; A61M 5/14566; A61M 2005/14252; A61M 2005/14506; A61M 2210/04

(57) ABSTRACT

A method of operating a medicament delivery device is provided. The method can include selecting a medicament delivery device, comprising: an external housing including a substantially rigid body; a moveable medicament container sled; a needle insertion mechanism; a fluid path operable to connect a needle of the needle insertion mechanism with a medicament container; a medicament container including a container body and plunger, wherein the medicament container is secured in a portion of the moveable medicament container sled; and a drive mechanism. The method can include applying the medicament delivery device to a skin surface of a patient; and activating the medicament delivery device to cause the drive mechanism to apply pressure to the plunger of the medicament container, wherein activating the medicament delivery device comprises releasing a latch mechanism of the drive mechanism.

The present disclosure relates generally to devices and methods for a large volume injection device, and more specifically for operating a large volume injection device.

Injection devices may be used to deliver a fluid containing a pharmaceutical drug or medicament to a patient. For example, the medicament may be delivered by the injection device to the patient via a needle, cannula, tube, microneedle array, or other route.

One type of injector used to provide such a medicament is a large volume device (LVD), which may also be known as a bolus injector or a reservoir-type injector. A large volume device may provide a relatively large volume of medicament, typically at least 1 mL or more. The large volume device is generally positioned against the skin or held to the skin at a suitable injection site, and upon activation of the device, medicament is injected through the patient's skin.

The medicament must be provided to the injection device and made accessible so that the injection device can deliver the medicament to the patient. The medicament will be provided in a vial or container, and invariably, the medicament is sterilized in the vial or container. The vial or container is loaded into the large volume delivery device, and when the device is ready to be used, a connection must be formed between a delivery flow path and the medicament in the container. However, assembling the device in a sufficiently clean (e.g., sterile) manner such that the final device including the medicament container is substantially free of microorganisms, can be complicated and expensive. Furthermore, the assembled device must have a mechanism to maintain sterility or prevent introduction of microorganisms, but also allow formation of a connection between the medicament container and fluid flow path. Accomplishing these goals is challenging, and current devices for large volume delivery have various drawbacks.

Accordingly, there remains a need for devices and methods to establish a fluid connection to a reservoir of medicament in an injection device in a sterile or aseptic manner.

### SUMMARY

According to certain embodiments, a method of operating a medicament delivery device is provided. The method can include selecting a medicament delivery device, comprising: an external housing including a substantially rigid body; a moveable medicament container sled; a needle insertion mechanism; a fluid path operable to connect a needle of the needle insertion mechanism with a medicament container; a medicament container including a container body and plunger, wherein the medicament container is secured in a portion of the moveable medicament container sled; and a drive mechanism. The method can include applying the medicament delivery device to a skin surface of a patient; and activating the medicament delivery device to cause the drive mechanism to apply pressure to the plunger of the medicament container, wherein activating the medicament delivery device comprises releasing a latch mechanism of the drive mechanism.

According to certain embodiments, a method of operating a medicament delivery device is provided. The method can include selecting a medicament delivery device, comprising: an external housing including a substantially rigid body; a moveable medicament container sled; a needle insertion mechanism; a fluid path operable to connect a needle of the needle insertion mechanism with a medicament container; a medicament container including a container body and plunger, wherein the medicament container is secured in a portion of the moveable medicament container sled; and a drive mechanism. The method can include applying the medicament delivery device to a skin surface of a patient; activating the medicament delivery device by substantially simultaneously: activating the needle insertion mechanism to cause the needle of the needle insertion mechanism to pierce the skin surface of the patient; and activating the drive mechanism to cause the drive mechanism to apply pressure to the plunger of the medicament container to cause the moveable medicament container sled to move, thereby advancing the medicament container and causing the container to become fluidly connected with the fluid path.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a schematic diagram of an exemplary medicament delivery device.

FIG. 2 is a perspective view of an exemplary delivery device.

FIG. 3 provides an exploded perspective view of a delivery device, according to various embodiments.

FIG. 4 is a schematic illustration of a method of assembling a device, according to certain embodiments.

FIG. 5 illustrates an aseptic fluid path connection, according to certain embodiments.

FIG. 6 illustrates a partially assembled perspective view to illustrate assembly, according to certain embodiments.

FIGS. 7A and 7B are side cut away views of a medicament delivery device illustrating operation of various components including a container sled.

FIGS. 8A and 8B are side cut away views of a medicament delivery device illustrating operation of various components including an activation mechanism.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purposes.

The presently discussed devices, methods, and systems can be used to load a medicament container into a large volume delivery device in a convenient and cost-effective manner while maintaining a high degree of sanitation for critical components.

The present disclosure is described as providing “aseptic” connections for drug delivery devices. As used herein, “aseptic” or “aseptic condition” will be understood to refer to a condition wherein a device is free of substantially all, but not necessarily all microorganisms such as bacteria, viruses or fungi. As used herein, an “aseptic connection” refers to components that can allow connection of a fluid flow path with a medicament container of a device while preventing introduction of microorganisms. The “connection” need not be already connected but can be capable of forming the connection when ready for use. The terms “aseptic,” “aseptic condition” or “aseptic connection” may not necessarily require that the device be sterile or free of all microorganisms (as sterile may be defined by regulatory requirements), but “aseptic” and “aseptic connection” will be understood to encompass devices that are sterile or maintain sterility. Further, terms “sterile,” “sterile condition,” “sterile connection,” “sterilizing,” or its variations will be understood to include descriptions for the terms “aseptic,” “aseptic condition” and “aseptic connection” and/or very nearly sterile conditions or processes, unless indicated otherwise. Use of the terms “sterile,” “sterile condition,” “sterile connection,” “sterilizing” or its variations will be understood to include use of the terms “aseptic,” “aseptic condition” and “aseptic connection” and/or very nearly sterile conditions or processes, unless indicated otherwise.

Typical injection volumes can range from about 1 mL to over 10 mL. The devices may produce a wide range of injection rates from 0.2 mL/min up to 204.0 mL/min. Such injection profiles may be generally constant in flow rate, generally continuous in duration, or both generally constant and generally continuous. These injections can also occur in a single step of administration. Such injection profiles may be referred to as bolus injections.

Delivery devices functioning with such medicaments may utilize a needle, cannula, or other injection element configured to deliver a medicament to the patient. Such an injection element may, for example, have an external size or diameter of 27G or less. Further, the injection element could be rigid, flexible, and formed using a range of one or more materials. And in some embodiments, the injection element may include two or more components. For example, a rigid trocar may operate in conjunction with a flexible cannula. Initially, both the trocar and cannula may move together to pierce the skin. The trocar may then retract while the cannula remains at least partially within the target tissue. Later, the cannula may separately retract into the delivery device.

An example drug delivery device may involve a needle-based injection system as described in ISO 11608-1:2022. Needle-based injection systems may be broadly distinguished into multi-dose container systems and single-dose (with partial or full evacuation) container systems. The container may be a replaceable container or an integrated non-replaceable container.

A multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2022, a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). A single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An insertion mechanism for inserting the needle may take any suitable form. It may be a mechanical spring-based mechanism. Alternatively, the insertion element mechanism may for instance include an electric motor and a gear mechanism that causes insertion of the insertion element into the user. Needle insertion may also be part of a manual action by a user achieved before medicament delivery starts. Alternatively, the insertion mechanism may be a gas or fluid pressure operated mechanism, in which case the needle driving energy source is either a reservoir of pressurized gas or a chemical system in which two or more chemicals are mixed together to produce gas or fluid pressure.

One type of delivery device includes a Large Volume Device (LVD). An LVD delivery device is configured to dispense a relatively large dose of medicament, in particular at least 1 mL and typically up to 2.5 mL, but possibly up to 10 mL. LVDs can also be configured for bolus or basal delivery.

A bolus LVD injector device is configured to deliver a bolus of the respective medicament to bring a volume of the medicament into a patient's body within a predetermined time. The injection rate, however, may not be critical, i.e., tight control may not be necessary. However, there may be an upper (physiological) limit to the delivery rate in order to avoid damage to the tissue surrounding the delivery site. The time taken to deliver a bolus dose of medicament may be between a few minutes and many hours depending on a number of factors including the quantity (volume) of medicament, the viscosity of the medicament and the nature of the injection site at which the injection device is intended to be used.

From a user or health care professional perspective, it is desirable for an injection device to be configured to minimally impact the patient's lifestyle and schedule, providing the patient with minimal reminder of his or her disease between the injections. The treatment schedule for therapies is usually intermittent, i.e. may be one injection per week, one injection every other week, or one per month. Therefore, the patient usually has no routine in dealing with his or her disease, and hence has minimal routine/experience in performing the required injections. Thus, configuration of the injection device to simplify its operation by patients is highly desirable.

If an LVD is intended for bolus operation, the configuration of the LVD injection device is quite different compared to an LVD injection device that is intended to be used for basal operation. Also, its use is quite different. For instance, a basal-type insulin pump generally is relatively expensive as it includes many sophisticated diabetes specific features like programmable delivery rate profiles, bolus calculators etc. Further, the connection to the body via an infusion set allows the patient to handle and manipulate the pump in his/her field of view while the therapy is ongoing. Further, diabetes patients usually have a routine in setting-up the infusion set, connecting and operating the pump, and disconnecting the pump temporarily for events like taking a shower so not to expose the pump to water. In contrast, the bolus injector devices described above can be relatively simple and inexpensive devices. They may be provided as single-use devices, which cannot be recharged with medicament, which further reduces complexity and cost.

To use an LVD injection device, it is first located on a suitable injection site on a patient's skin. The device is typically adhered to the patient's skin throughout the medicament delivery process. Injection is usually initiated by the patient or another person (user). Typically, the initiation is started by a user operation, such as depressing a switch (mechanical or electrical) or by placing the LVD on the patient's body and depressing a lever on the device's underside. If the LVD includes electronics, a controller can operate the device. Operation includes firstly injecting a needle into the user and then causing the injection of medicament into the user's tissue. The delivery process can take several minutes up to several hours. Following, the LVD can be removed from the injection site and disposed of.

Biological medicaments are being increasingly developed which comprise higher viscosity injectable liquids and which are to be administered in larger volumes than long-known liquid medicaments. LVDs for administering such biological medicaments may comprise a pre-filled disposable drug delivery device or, alternatively, a disposable drug delivery device into which a patient or medical personnel must insert a drug cartridge prior to use.

In some embodiments, medicaments of various viscosities can be injected. For example, viscosity could range from about 3 to about 50 cP. In other embodiments, viscosity could be less than about 3 cP or greater than about 50 cP. Injection can further include delivering a medicament to a sub-cutaneous, an intra-muscular, or a transdermal location within a patient's body. The medicament can be in the form of a liquid, gel, slurry, suspension, particle, powder, or other type.

In some embodiments, the large volume devices may include a housing configured to be held against the user's body when the device is in use, a medicament cartridge, a fluid pathway connected to the medicament cartridge and extending to an insertion mechanism, such as a needle insertion mechanism or a trocar and cannula insertion mechanism. The medicament may be drawn or forced from the medicament cartridge by any method, such as by a plunger mechanism or by a pump, and injected into the user by the same force, or by a separate plunger or pump. In some embodiments, the plunger may be driven by one or more springs, drive screws, motors, or any other suitable drive mechanism.

The terms “drug”, “medicament”, or “pharmaceutical”, which are used interchangeably herein, mean a pharmaceutical formulation that includes at least one pharmaceutically active compound, which may be, for example, a small molecule or biologic active pharmaceutical ingredient. Further descriptions of contemplated drugs, medicaments or pharmaceuticals are provided below.

Standards or best practices for the design of drug delivery devices may require or recommend providing an unobstructed path from the medicament container to a needle or cannula used to deliver the medicament to a user. Additionally, standards or best practices for manufacturing and assembling a drug-delivery device, such as a large volume or reservoir-type injection device, may require that certain steps in the assembly occur in a clean room or aseptic facility or may require sanitizing certain components. Embodiments may provide an aseptic sterile path from a drug container to the needle or cannula, according to the present disclosure, without requiring that all aspects of the assembly be performed in clean room or highly sterilized environments. Embodiments may provide flexibility and cost-savings in manufacturing by allowing aseptic conditions to be maintained in the device without requiring the use of a clean room or other highly sanitized environment during all steps of the manufacturing process. For example, the medicament container may be inserted into the injection device outside of a clean room or highly sanitized environment while still providing an aseptic connection. Another advantage is that the manufacture of the injection device and the loading of the injection device with a medicament container may be performed independently and the reservoir may be added in a wider range of facilities. For example, the reservoir may be supplied in a separate manufacturing process or may be supplied by a medical technician, compounding pharmacy, or a user without requiring a clean room or highly sterilized environment.

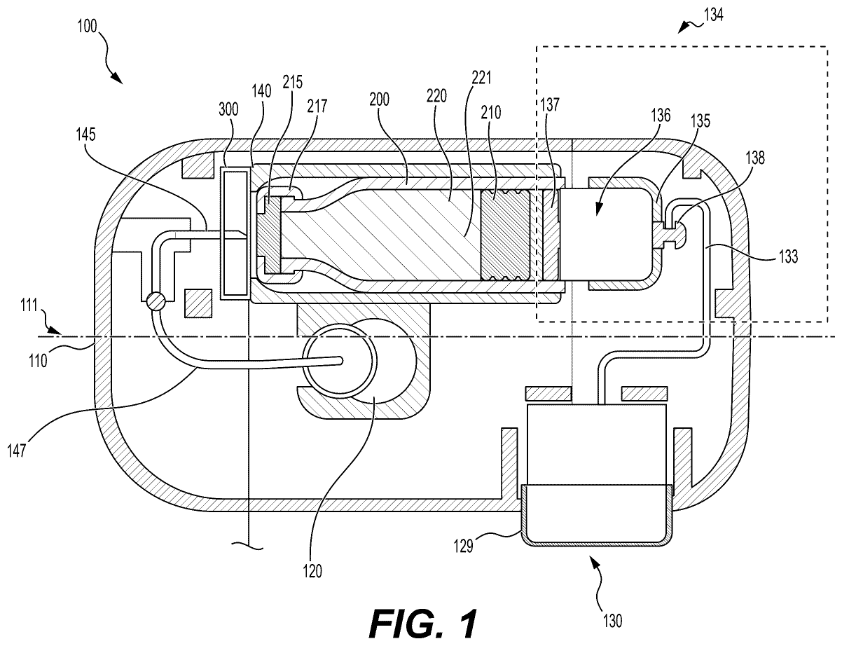

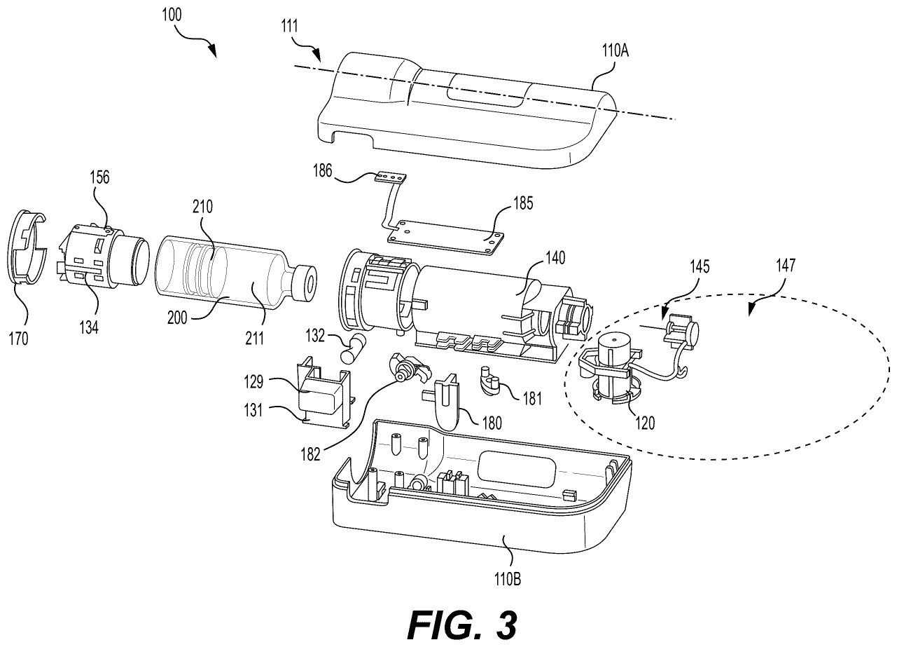

FIG. 1 illustrates an exemplary large volume delivery device 100 with a housing 110, a needle insertion mechanism 120, a release mechanism 130, a drive mechanism 134, a cartridge holder 140, which can be in the form of a moveable medicament container sled 140, a needle 145, and a fluid path 147. The release mechanism 130 includes a button 129, a button sled 131 (as shown in FIG. 3) and a button biasing member or other power source (not shown). The button 129 is connected to the button sled 131 (as shown in FIG. 1B). In some embodiments, the button 129 is part of the button sled 131. The drive mechanism 134 includes a drive housing 135, a drive release latch 133, a piston mechanism including a piston 137, a drive outer sleeve 136, and a detent 138.

A medicament container 200 is included within the large volume delivery device 100. The medicament container 200 includes a plunger 210, a stopper 215 (which may alternatively be referred to as a septum), and a cap 217 (e.g., a crimp cap) and has an internal volume 220 at least partially filled with a medicament 221. The container 200 may be, for example, a glass vial with a polymer plunger and septum. Generally, the container 200 will include a standard medicament container such that the disclosed devices and fluid path connections can be use with existing, standard containers without the need for development of a specialized container or medicament cartridge. The plunger 210 may be driven by the piston 137.

FIG. 1 should be understood to be an exemplary device. The drive mechanism 134 can include one or more springs, but other drive mechanisms or power sources may be possible. Other drive mechanisms may include other biasing elements, screw drives, gear drives, gas or chemical sources, or electric motors. The medicament container 200 should be understood as exemplary only. For example, different types of containers or vials may be used, including containers with different stopper or piston arrangements, or without a stopper or piston.

The large volume delivery device 100 is prepared for use by loading the medicament container 200 and setting the drive mechanism 134 in the energized state (as shown in FIG. 1). The drive outer sleeve 136 is held in an energized state by engagement of the drive release latch 133 with a detent 138 of the piston 137. To operate the large volume delivery device 100, the large volume delivery device 100 is positioned with the needle insertion mechanism 120 against skin of a user. The large volume device delivery 100 may be attached to skin of the user by removing a removable covering mounted on the external housing 110 to expose an adhesive on the external housing 110. The user, or someone assisting the user, presses the button 129 and/or the button sled 131 of the release mechanism 130 to shift the drive release latch 133 out of the detent 138 and release the drive outer sleeve 136, whereupon the drive outer sleeve 136 drives the piston 137 against the plunger 210 of the medicament container 200, forcing the plunger 210 into the medicament container 200 and pressurizing the internal volume 220 with medicament 221. An action of the large volume delivery device 100 causes the needle 145 to pierce the stopper 215 and access the medicament 221 within the medicament container 200. The pressurized medicament flows from the medicament container 200 into the needle 145 and then along the fluid path 147 to the needle insertion mechanism 120. An action of the large volume device delivery 100 causes the needle insertion mechanism 120 to insert a needle and/or cannula into the user. The medicament 221 is delivered through the needle or cannula to the patient.

The large volume delivery device 100 also includes a fluid path connection 300. The fluid path connection 300 is positioned near the stopper 215 and needle 145 and permits the needle 145 to travel through an unobstructed path to the stopper 215 to establish a fluid path connection with the medicament container 200. In some embodiments, the fluid path connection 300 is connection is configured to isolate the fluid path during storage and/or use and to maintain an aseptic or sterile fluid path.

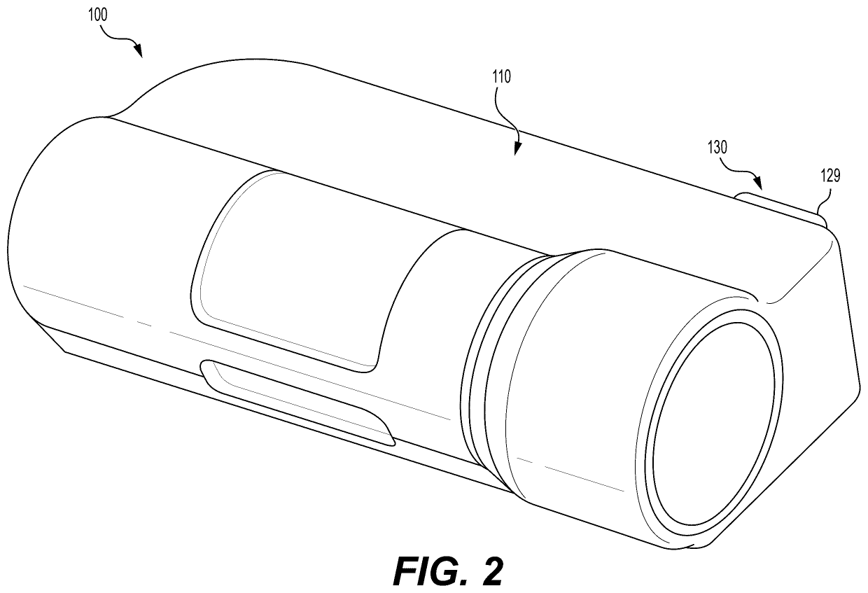

FIG. 2 provides a perspective view of a large volume delivery device 100 (also referred to as “device 100”) having a more specific shape, but which can include some or all of the same components described with respect to FIG. 1. The device 100 of FIG. 2 includes the external housing 110, as well as a button 129 that acts as the release mechanism 130 for the drive mechanism 134 (as shown in FIG. 1). As described further below, the button 129 and/or the button sled 131 is placed laterally or towards the side of the delivery device 100 to allow application of pressure orthogonal to an axis of the drive mechanism 134 (as shown in FIG. 1). As such, the button 129 and/or the button sled 131 can apply lateral or sideways directed force to a latch or securing mechanism of the drive mechanism 134 (as shown in FIG. 1), and optionally activate one or more additional components of the device substantially simultaneously with the drive mechanism 134 (as shown in FIG. 1).

FIG. 3 illustrates the exemplary large volume delivery device 100 and its assembly from a perspective view. As shown in FIG. 3, the exemplary device 100 includes the external housing 110 that includes the medicament container sled 140 and a drive holder 160, a medicament container 200 that includes a plunger 210 and an internal volume 220 to hold medicament, the drive mechanism 134, an end of dose (EOD) switch assembly 156, and a housing drive cap 170.

As shown in FIG. 3, the external housing 110 of the exemplary device 100 further includes a top cover 110A and a bottom cover 110B, an interlock fin 180, and interlock with an interlock spring 182, the release mechanism 130 including a button 129, a button sled 131 and a button spring 132, and a needle insertion mechanism (NIM) lever 181 and a fluid path assembly 147. Additionally, the housing 110 includes an axis 111, along which the medicament container 200 moves via movement of the medicament container sled 140.

In some embodiments, the medicament container 200 may be configured to enter the medicament container sled 140, and may be configured to retained into the medicament container sled 140 with a secure connection. For example, the container 200 may be secured by at least at least one snap-fit connector on the medicament container sled 140 or by a friction fit or other connection.

In some embodiments, the drive mechanism 134 may be configured to enter the drive holder 160, and may be configure to be retained into the drive holder 160. For example, the drive mechanism may be secured by at least one snap fit connector on the drive holder 160 and/or at least one mechanical stop.

In some embodiments, the drive mechanism 134 may be configured to interact with the EOD switch assembly 156 to detect nearing of end of dose.

As explained above, a method of operating a medicament delivery device is provided. The method can include selecting a medicament delivery device 100, comprising: an external housing 110 including a substantially rigid body; a moveable medicament container sled 140; a needle insertion mechanism 120; a fluid path 147 operable to connect a needle of the needle insertion mechanism 120 with a medicament container 200; a medicament container 200 including a container body and plunger 210, wherein the medicament container 200 is secured in a portion of the moveable medicament container sled 140 ; and a drive mechanism 134. The method can include applying the device 100 to a skin surface of a patient; and activating the delivery device to cause the drive mechanism 134 to apply pressure to the plunger 210 of the medicament container, wherein activating the delivery device 100 comprises releasing a latch mechanism 133 of the drive mechanism 134.

According to certain embodiments, a method of operating a medicament delivery device is provided. The method can include selecting a medicament delivery device 100, comprising: an external housing 110 including a substantially rigid body; a moveable medicament container sled 140; a needle insertion mechanism 120; a fluid path 147 operable to connect a needle of the needle insertion mechanism 120 with a medicament container 120; a medicament container including a container body and plunger 210, wherein the medicament container 200 is secured in a portion of the moveable medicament container sled 140; and a drive mechanism 134. The method can include applying the device to a skin surface of a patient; activating the delivery device 100 by substantially simultaneously: activating the needle insertion mechanism 120 to cause the needle 145 of the needle insertion mechanism 120 to pierce the skin surface of the patient; and activating the drive mechanism 134 to cause the drive mechanism 134 to apply pressure to the plunger 210 of the medicament container 200 to cause the moveable medicament container sled 140 to move, thereby advancing the medicament container 200 and causing the medicament container 200 to become fluidly connected with the fluid path 147.

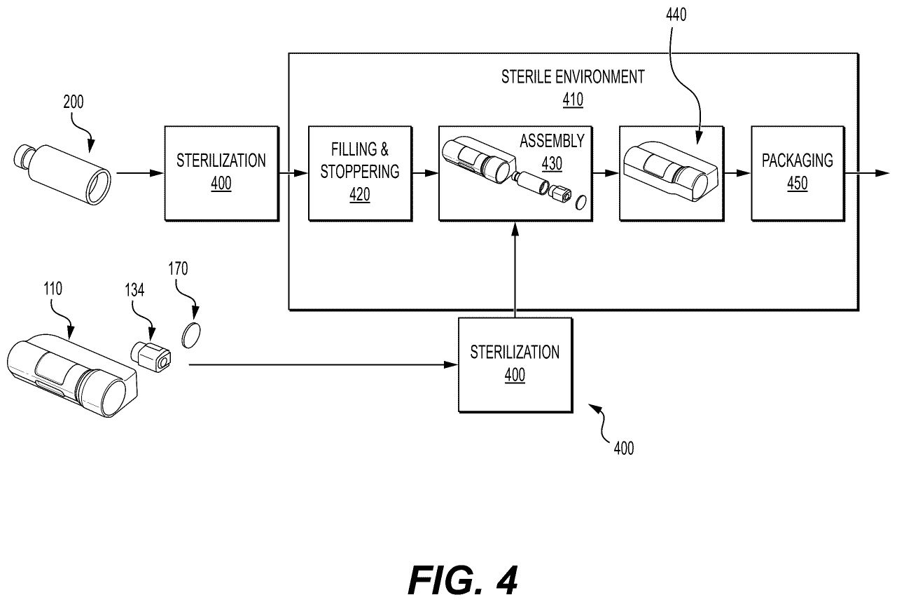

FIG. 4 provides a schematic diagram of a method of assembly, according to certain embodiments. As shown, the method can include selection of the medicament container components 200 and the delivery device components 100. It will be understood that the container components 200 and delivery device components 100 can include the various components described with respect to FIG. 1, including, but not limited to, an exemplary large volume device 100 with an external housing 110, a needle insertion mechanism 120, a release mechanism 130, a drive mechanism 134, a medicament container sled 140, a needle 145, and a fluid path 147 for the device 100; and for the container 200, a plunger 210, a stopper 215 (which may alternatively be referred to as a septum), and a cap 217 (e.g., a crimp cap). Variations on the device 100 or container 200 will be understood to be feasible.

The delivery device 100 and/or container 200 may be received with all components separated or partially assembled. For example, as shown, the device 100 can be assembled to include all internal components except for the medicament container 200, drive mechanism 134 and housing drive cap 170. But it will be understood that the device 100 and container 200 can be received at various levels of assembly.

The device 100 and container 200 can be sent to a sterilization module 400 where the devices are sterilized. A number of conventional sterilization methods can be used such as chemical, thermal, or radiation methods (e.g., e-beam, gamma). The method can therefore include sterilizing the medicament delivery device main body, the drive mechanism, and the drive housing cap; sterilizing the medicament container.

After sterilizing the components, under sterile conditions, the method can include assembling the device 100 by placing the medicament container 200 in the medicament container sled 140 (Module 430). In some cases, the container 200 will be pre-filled with medicament and sterilized as such. Alternatively, the container 200 will be filled (Module 420) after sterilizing, and the method will include filling and stoppering the container 200 under sterile conditions. After assembling the device 100, a final closure can be performed by applying the housing drive cap 170 and performing final inspection (Module 440). Finally, finally packaging can be performed (Module 450) to provide a sterile barrier, add instructions for use and labels, and prepare for shipping and storage.

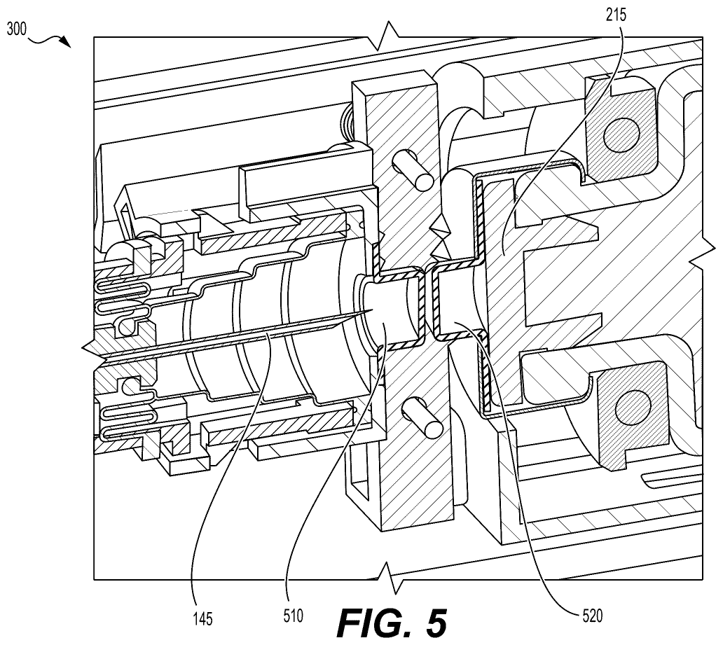

In some embodiments, the method includes placing the medicament container 200 in the medicament container sled 140 under sterile or aseptic conditions. Further, this step can also include forming an aseptic region between a cap of the medicament container and the fluid path. The aseptic region can include the fluid path connection 300 illustrated in FIG. 1. This fluid path connection 300 can include a first sterile barrier 510 over a cap of the medicament container 200 and a second sterile barrier 520 over a piercing member of the fluid path (FIG. 5). As shown, the barriers 510, 520 can include caps, but the barriers 510, 520 could be at least one of a cap, a foil, or a film. These barriers 510, 520 can be formed before or after sterilization but before assembly of the final device by inserting the container 200 into the medicament container sled 140 of the device 100.

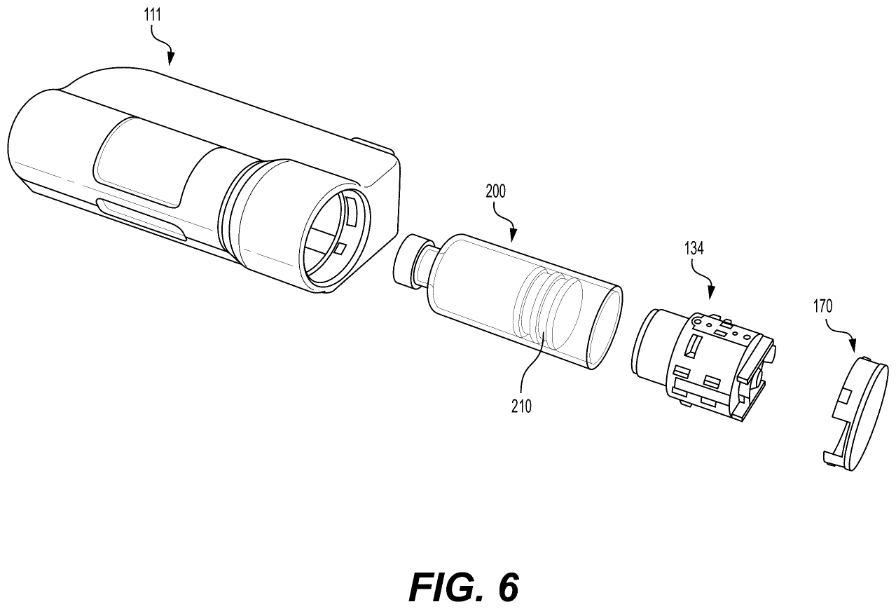

The method of assembling the device 100 can further include individual steps under sterile conditions or before sterilization. For example, as shown in FIG. 6, the method can include placing the drive mechanism 134 proximate the plunger 210 of the medicament container 200, and securing the housing drive cap 170 proximate a rear portion of the drive mechanism 134 to close the device 100.

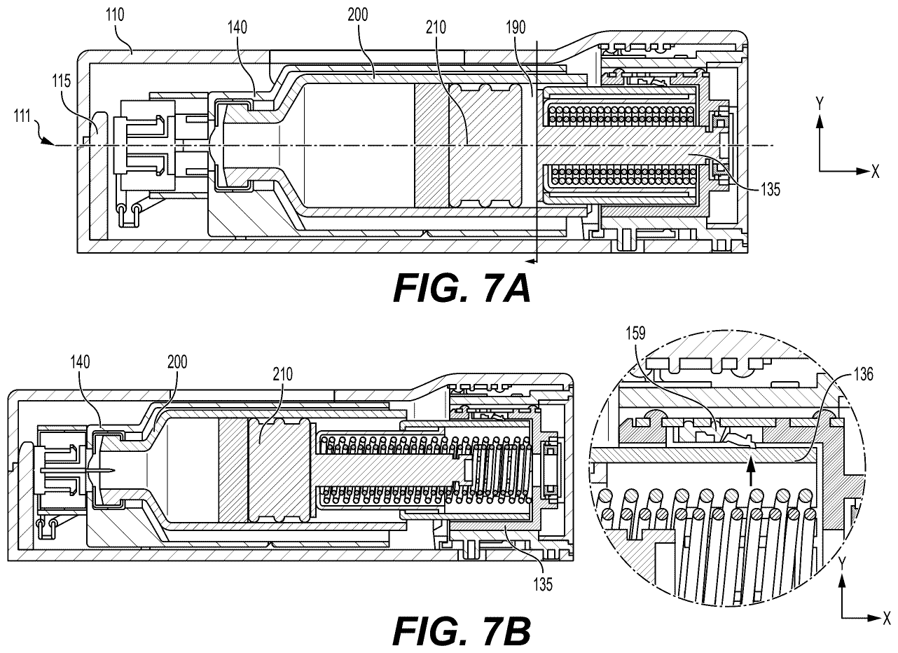

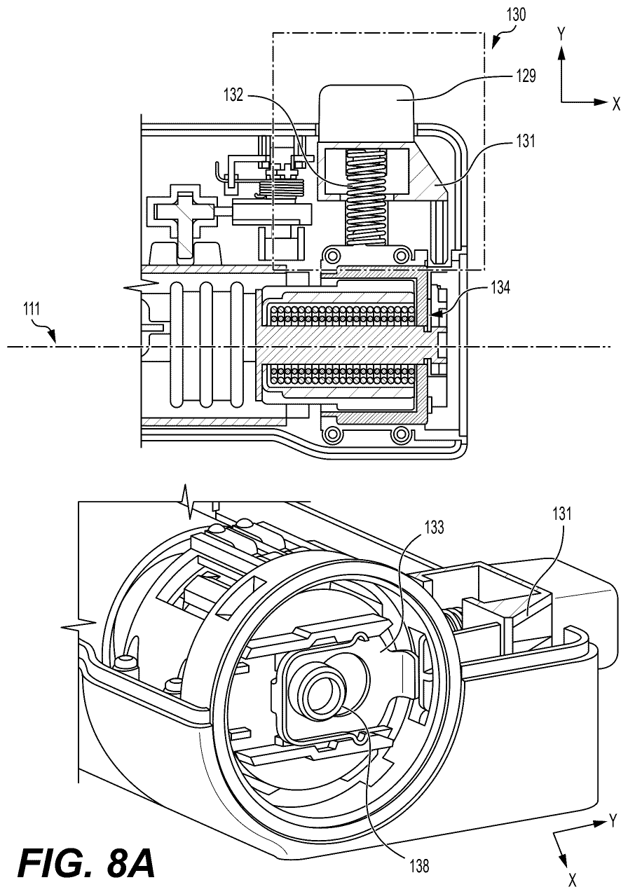

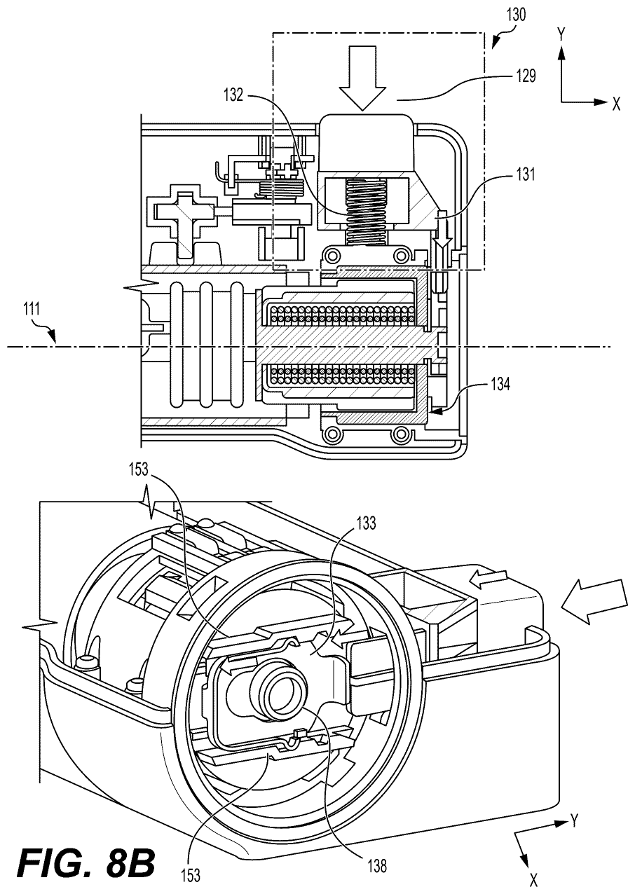

FIGS. 7A, 7B, 8A, and 8B further illustrate aspects of the method of operation. FIGS. 7A and 8A illustrates a configuration before activation or operation of the device 100. As shown, before activation in FIG. 7A, the moveable medicament container sled 140 is located such that the medicament container 200 is not pierced by a needle 145 of the fluid path 147. But, upon activation, the drive mechanism 134 causes the medicament container sled 140 to move forward (FIG. 7B), thereby moving the medicament container sled 140 and the medicament container 200, such that the medicament container 200 is pierced by the needle 145. In some cases, the movement of the medicament container sled 140 is initiated substantially simultaneously with activation of the needle insertion mechanism 120.

FIGS. 8A and 8B illustrate an exemplary release system 130 that may be used to activate the drive mechanism 134 and or other components of the device such as the needle insertion mechanism 120. As shown, the release mechanism 130 can include a drive release latch 133 connected to the drive detent 138.

The drive release latch 133 may move laterally or substantially orthogonal to an axis 111 of the medicament container 200 and drive mechanism 134 by movement or force from the button 129 and/or the button sled 131 of the release mechanism 130.

In some cases, the medicament container sled 140 may move in such a way as to cause release of the needle insertion mechanism. In other embodiments, the button 129 and/or the button sled 131 may be directly mechanically connected to the needle insertion mechanism 120. Further, the needle insertion mechanism 120 may be activated independently of the button sled 131 and the medicament container sled 140.

As shown in FIG. 8A, upon that the drive mechanism 134 is inserted and retained in the drive holder 160, the drive release latch 133 may contact with a button 129 and/or button sled 131 of the release mechanism 130. In some embodiments, the drive release latch 133 may be released and move along the pair of walls 153 to unlock the detent 138 upon receiving pressure from the button 129 and/or button sled 131. Specifically, as shown in FIG. 8B, pressure is applied to the button 129 and/or button sled 131 of the release mechanism 130, for example, by the user, the button 129 and/or button sled 131 is shifted to move along an Y-axis (which is perpendicular to the axis 111 of the external housing 110) and pushes the drive release latch 133 to move along between the pair of walls 153 from the initial position (shown in FIG. 8A) to the activated position (shown in FIG. 8B) to release the detent 138. Once the detent 138 is released by the drive release latch 133, the piston 137 is released.

### Exemplary Drugs or Medicaments

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA (including RNAi & siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In other examples, the container may be made of a flexible elastomeric material and designed to be loaded by the health care provider or patient, then placed on the body for administration. In some instances, the chamber may be designed to store a drug for about 1 month to about 2 years. Storage may occur at room temperature (e.g., about 20° C.), or refrigerated temperatures (e.g., from about −4°C to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423,NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864,ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is

1. A method of operating a medicament delivery device, comprising:
selecting a medicament delivery device, comprising:
an external housing including a substantially rigid body;
a moveable medicament container sled;
a needle insertion mechanism;
a fluid path operable to connect a needle of the needle insertion mechanism with a medicament container;
a medicament container including a container body and plunger, wherein the medicament container is secured in a portion of the moveable medicament container sled; and
a drive mechanism;
applying the medicament delivery device to a skin surface of a patient; and
activating the medicament delivery device to cause the drive mechanism to apply pressure to the plunger of the medicament container to cause the moveable medicament container sled to move, thereby advancing the medicament container and causing the medicament container to become fluidly connected with the fluid path.

2. The method of claim 1, wherein activating the medicament delivery device comprises releasing a latch mechanism of the drive mechanism.

3. The method of claim 2, wherein releasing the latch mechanism comprises pushing a button on a side portion of the external housing to push the latch mechanism substantially orthogonally to an axis of the drive mechanism.

4. The method of claim 1, wherein the medicament delivery device comprises an aseptic region between a cap of the medicament container and the fluid path, and the method further includes disrupting at least one barrier of the aseptic region to provide a path for connecting the medicament container with the fluid path.

5. The method of claim 4, wherein the at least one barrier includes a first barrier covering a cap of the medicament container and a second barrier covering a portion of the fluid path.

6. The method of claim 5, wherein the first barrier and the sterile barrier each comprise at least one of a cap, a foil, or a film.

7. The method of claim 1, wherein activating the medicament delivery device further comprises activating the needle insertion mechanism to cause the needle of the needle insertion mechanism to pierce the skin surface of the patient.

8. The method of claim 1, wherein causing the medicament container to become fluidly connected with the fluid path includes causing a needle in communication with the fluid path to pierce a cap portion of the medicament container as the medicament container is advanced.

9. The method of claim 1, wherein the moveable medicament container sled is secured by a mechanical stop, and activating the medicament delivery device comprises causing the mechanical stop to be released from the moveable medicament container sled.

10. A method of operating a medicament delivery device, comprising:
selecting a medicament delivery device, comprising:
an external housing including a substantially rigid body;
a moveable medicament container sled;
a needle insertion mechanism;
a fluid path operable to connect a needle of the needle insertion mechanism with a medicament container;
a medicament container including a container body and plunger, wherein the medicament container is secured in a portion of the moveable medicament container sled; and
a drive mechanism;
applying the device to a skin surface of a patient;
activating the delivery device to cause the drive mechanism to apply pressure to the plunger of the medicament container, wherein activating the medicament delivery device comprises releasing a latch mechanism of the drive mechanism.

11. The method of claim 10, wherein releasing the latch mechanism comprises pushing a button on a side portion of the external housing to push the latch mechanism substantially orthogonally to an axis of the drive mechanism.

12. The method of claim 10, wherein the delivery device comprises an aseptic region between a cap of the medicament container and the fluid path, and the method further includes disrupting at least one barrier of the aseptic region to provide a path for connection the medicament container with the fluid path.

13. The method of claim 12, wherein the at least one barrier includes a first barrier covering a cap of the medicament container and a second barrier covering a portion of the fluid path.

14. The method of claim 13, wherein the first barrier and the sterile barrier each comprise at least one of a cap, a foil, or a film.

15. The method of claim 10, wherein activating the medicament delivery device further comprises activating the needle insertion mechanism to cause the needle of the needle insertion mechanism to pierce the skin surface of the patient.

16. The method of claim 10, wherein causing the medicament container to become fluidly connected with the fluid path includes causing a needle in communication with the fluid path to pierce a cap portion of the medicament container as the medicament container is advanced.

17. A method of operating a medicament delivery device, comprising:
selecting a medicament delivery device, comprising:
an external housing including a substantially rigid body;
a moveable medicament container sled;
a needle insertion mechanism;
a fluid path operable to connect a needle of the needle insertion mechanism with a medicament container;
a medicament container including a container body and plunger, wherein the medicament container is secured in a portion of the moveable container sled; and
a drive mechanism;
applying the device to a skin surface of a patient;
activating the delivery device by substantially simultaneously:
activating the needle insertion mechanism to cause the needle of the needle insertion mechanism to pierce the skin surface of the patient; and
activating the drive mechanism to cause the drive mechanism to apply pressure to the plunger of the medicament container to cause the moveable medicament container sled to move, thereby advancing the medicament container and causing the medicament container to become fluidly connected with the fluid path.

18. The method of claim 17, wherein activating the medicament delivery device comprises releasing a latch mechanism of the drive mechanism.

19. The method of claim 18, wherein releasing the latch mechanism comprises pushing a button on a side portion of the external housing to push the latch mechanism substantially orthogonally to an axis of the drive mechanism.

20. The method of claim 17, wherein the medicament delivery device comprises an aseptic region between a cap of the medicament container and the fluid path, and the method further includes disrupting at least one barrier of the aseptic region to provide a path for connection the medicament container with the fluid path.

21. The method of claim 20, wherein the at least one barrier includes a first barrier covering a cap of the medicament container and a second barrier covering a portion of the fluid path.

22. The method of claim 21, wherein the first sterile barrier and the second sterile barrier each comprise at least one of a cap, a foil, or a film.

23. The method of claim 17, wherein causing the medicament container to become fluidly connected with the fluid path includes causing a needle in communication with the fluid path to pierce a cap portion of the medicament container as the medicament container is advanced.

24. The method of claim 17, wherein the moveable medicament container sled is secured by a mechanical stop, and activating the delivery device comprises causing the mechanical stop to be released from the moveable medicament container sled.
